(19) United States
(12) Patent Application Publication
(10) Pub. No.: US 2023/0256033 A1
(43) Pub. Date: Aug. 17, 2023

(54) PHYSIOLOGICALLY ACCEPTABLE COMPOSITIONS CONTAINING MICROORGANISMS OR MICROBIAL PRODUCTS

(71) Applicant: FLAGSHIP PIONEERING INNOVATIONS VI, LLC, Cambridge, MA (US)

(72) Inventors: Douglas Gowers COLE, Wayland, MA (US); Jason PARK, West Newton, MA (US); Sabrina Yusang YANG, Cambridge, MA (US); Andrew John GASPARRINI, Boston, MA (US)

(21) Appl. No.: 18/009,395

(22) PCT Filed: Jun. 9, 2021
(86) PCT No.: PCT/US21/36622

§ 371 Date: Dec. 9, 2022

Related U.S. Application Data
(60) Provisional application No. 63/036,543, filed on Jun. 9, 2020.
(60) Provisional application No. 63/140,493, filed on Jan. 22, 2021.

(51) Int. Cl. A61K 35/74; C12N 1/20; A61P 29/00

(52) U.S. Cl. CPC A61K 35/74; C12N 1/20; A61P 29/00

(57) ABSTRACT

The invention relates to physiologically acceptable compositions of isolated microorganisms, lysates thereof, and supernatants therefrom, as well as methods of their use and preparation.

### FIELD OF THE INVENTION

The invention relates to physiologically acceptable compositions of isolated microorganisms, lysates thereof, and supernatants therefrom, as well as methods of their use and preparation.

### BACKGROUND

Mammals are colonized by microorganisms in the gastrointestinal (GI) tract, on the skin, and in other epithelial and tissue niches. The gastrointestinal tract of a healthy individual harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth and is believed to form a functionally mature microbial population by about 3 years of age. Interactions between microbial strains in these populations and between microorganisms and the host, e.g., interactions with the host's immune system, shape the community structure, with availability of and competition for resources affecting the distribution of microorganisms.

A healthy microbiome may provide a subject with multiple benefits, including colonization resistance to a broad spectrum of pathogens, essential nutrient biosynthesis and absorption, and immune stimulation that plays a role in maintaining a healthy gut epithelium and appropriately controlled systemic immunity.

There is a need for compositions and methods for addressing problems in healthcare through controlling the microbiome.

### SUMMARY OF THE INVENTION

In one aspect, the invention provides a method of treating or preventing an inflammatory disease or condition in a subject in need thereof, the method comprising administering to the subject a therapeutically effective amount of a composition comprising an isolated microorganism of Table 1, a lysate thereof, or a supernatant thereof.

In some embodiments of the above aspect, the inflammatory disease or condition is an inflammatory bowel disease (IBD). In some embodiments, the IBD is ulcerative colitis (UC). In other embodiments, the IBD is Crohn's disease.

In some embodiments, the inflammatory disease or condition is an autoimmune disease or an allergy.

In some embodiments, the inflammatory disease or condition is selected from the group consisting of asthma, rheumatoid arthritis, dermatitis, psoriasis, psoriatic arthritis, multiple sclerosis, celiac disease, glomerulonephritis, hepatitis, and transplant rejection.

In another aspect, the invention provides a method of treating or preventing a disease or condition modulated by an inflammatory pathway in a subject in need thereof, the method comprising administering to the subject a therapeutically effective amount of a composition comprising an isolated microorganism of Table 1, a lysate thereof, or a supernatant thereof.

In some embodiments of the above aspect, the inflammatory pathway is an NFκB pathway or a TNFα pathway. In some embodiments, the NFκB pathway is an LPS-driven NFκB pathway or a TNFα-driven NFκB pathway.

In some embodiments, activation of the inflammatory pathway is decreased by at least 20% relative to a baseline level in the treated subject or relative to a subject who has not been treated with the composition.

In some embodiments, the disease or condition is an IBD. In some embodiments, the IBD is UC or Crohn's disease.

In another aspect, the invention provides a method of modulating an inflammation marker in a subject in need thereof, the method comprising administering to the subject a therapeutically effective amount of a composition comprising an isolated microorganism of Table 1, a lysate thereof, or a supernatant thereof.

In some embodiments, the inflammation marker is associated with an NFκB pathway or a TNFα pathway.

In some embodiments, the inflammation marker is NFκB, TNFα, TGFβ, IFNγ, IL12p40, IL-2, IL-4, IL-5, IL-6, IL-10, IL-13, IL-17, or a combination thereof.

In some embodiments, the inflammation marker is IL1β, IL8, IL17A, or a combination thereof.

In some embodiments, the inflammation marker is MCP-1, CCL20, NFAT, regulatory T cells (Tregs), Th17 induction, IL-23R, or IL-17A/F or a combination thereof.

In some embodiments, the inflammation marker is M2-PK pyruvate kinase, osteoprotegerin, MPO, HMGB1, CHI3L1, HBD2, MMP, calprotectin, lactoferrin, pANCA, ASCA, or a combination thereof.

In some embodiments, the inflammation marker is SAA, eotaxin-1, or a combination thereof.

In some embodiments, the modulation is a decrease in the level of the inflammation marker. In some embodiments, the decrease is at least 20% relative to a baseline level in the treated subject or relative to a subject who has not been treated with the composition.

In some embodiments, the modulation is an increase in the level of the inflammation marker.

In some embodiments, the composition comprises at least two isolated microorganisms of Table 1, lysates thereof, or supernatants thereof. In some embodiments, the composition comprises at least 3, 4, 5, 6, 7, 8, 9, 10, 15, 20, 25, 30, 35, 40, or 45 isolated microorganisms of Table 1, lysates thereof, or supernatants thereof.

In some embodiments, the composition comprises one or more of said isolated microorganisms. In some embodiments, the one or more isolated microorganisms colonizes the gut of the subject.

In some embodiments, the composition comprises a lysate of one or more of said isolated microorganisms. In some embodiments, the composition comprises a supernatant of one or more of said isolated microorganisms.

In some embodiments, the isolated microorganism was grown in a culture medium selected from the group consisting of BHI media, BHI-HK media, and BRU-AS media.

In some embodiments, (a) the isolated microorganism is microbe-005, microbe-056, or microbe-059 and the culture medium was BHI media; (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or (c) the isolated microorganism is microbe-041, microbe-043, microbe-045, or microbe-050 and the culture medium was BRU-AS media.

In some embodiments, the composition has been processed to remove endotoxin. In some embodiments, the composition has been sterilized.

In some embodiments, the composition has been processed to remove all components having a molecular weight of more than 3 kDa. In other embodiments, the composition has been processed to remove all components having a molecular weight of less than 3 kDa.

In another aspect, the invention provides a composition comprising an isolated microorganism of Table 1, a lysate thereof, or a supernatant thereof, wherein the composition is formulated as pharmaceutically acceptable composition, a comestible composition, or a nutraceutical.

In another aspect, the invention provides a composition comprising at least two isolated microorganisms of Table 1, lysates thereof, or supernatants thereof.

In some embodiments of the above two aspects, the composition is formulated as a physiologically acceptable powder, granule, capsule, or tablet.

In some embodiments, the composition is a pharmaceutical composition. In some embodiments, the composition is a comestible composition. In some embodiments, the composition is a nutraceutical. In some embodiments, the pharmaceutical composition is formulated for oral, enteral, or rectal administration.

In some embodiments, the composition comprises at least 3, 4, 5, 6, 7, 8, 9, 10, 15, 20, 25, 30, 35, 40, or 45 isolated microorganisms of any one or more of Table 1, lysates thereof, or supernatants thereof.

In some embodiments, the composition comprises one or more of said isolated microorganisms. In some embodiments, the one or more isolated microorganisms colonizes the gut of a subject.

In some embodiments, the composition comprises a lysate of one or more of said isolated microorganisms. In some embodiments, the composition comprises a supernatant of one or more of said isolated microorganisms.

In some embodiments, the isolated microorganism was grown in a culture medium selected from the group consisting of BHI media, BHI-HK media, and BRU-AS media.

In some embodiments, (a) the isolated microorganism is microbe-005, microbe-056, or microbe-059 and the culture medium was BHI media; (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or (c) the isolated microorganism is microbe-041, microbe-043, microbe-045, or microbe-050 and the culture medium was BRU-AS media.

In some embodiments, the composition has been processed to remove endotoxin. In some embodiments, the composition has been sterilized.

In some embodiments, the composition has been processed to remove all components having a molecular weight of more than 3 kDa. In some embodiments, the composition has been processed to remove all components having a molecular weight of less than 3 kDa.

In another aspect, the invention provides a method of growing an isolated microorganism of Table 1, the method comprising culturing the isolated microorganism in a culture medium selected from the group consisting of BHI media, BHI-HK media, and BRU-AS media.

In some embodiments, (a) the isolated microorganism is microbe-005, microbe-056, or microbe-059 and the culture medium is BHI media; (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium is BHI-HK media; or (c) the isolated microorganism is microbe-041, microbe-043, microbe-045, or microbe-050 and the culture medium is BRU-AS media.

In another aspect, the invention provides a composition described herein for use in a method of treating or preventing an inflammatory disease or condition in a subject in need thereof, wherein optionally the inflammatory disease or condition is an inflammatory bowel disease (IBD) (e.g., ulcerative colitis (UC) or Crohn's disease), is an autoimmune disease or an allergy, or is selected from the group consisting of asthma, rheumatoid arthritis, dermatitis, psoriasis, psoriatic arthritis, multiple sclerosis, celiac disease, glomerulonephritis, hepatitis, and transplant rejection.

In another aspect, the invention provides a composition described herein for use in a method of treating or preventing a disease or condition modulated by an inflammatory pathway in a subject in need thereof, wherein optionally the inflammatory pathway is an NFκB pathway or a TNFα pathway, wherein further optionally the NFκB pathway is an LPS-driven NFκB pathway or a TNFα-driven NFκB pathway, and further wherein optionally activation of the inflammatory pathway is decreased by at least 20% relative to a baseline level in the treated subject or relative to a subject who has not been treated with the composition.

In another aspect, the invention provides a composition described herein for use in a method of modulating an inflammation marker in a subject in need thereof. In some embodiments, one or more of the markers are as described above or elsewhere herein.

### BRIEF DESCRIPTION OF THE DRAWINGS

FIG. 1 is a bar graph showing activity of NFκB in the human monocyte cell line THP-1-DUAL™ cells (InvivoGen) challenged with LPS in the presence of supernatants from microbes 004, 005, 006, 009, 014, and 017 (Table 1). NFκB activity is shown as a percentage relative to a THP-1+LPS control. A microbe medium control is also shown. IKKi (IKK complex inhibitor) (2 μm), dexamethasone (10 μm), and cyclosporin (10 μm) are provided as control anti-inflammatory agents. Asterisks indicate significant differences, as assessed by ANOVA with Tukey's multiple comparisons.

FIG. 2A is a set of bar graphs showing the level of TNFα in LPS-stimulated human peripheral blood mononuclear cells (PBMCs) from three donors (Donor C, Donor D, and Donor E) treated with a supernatant from microbe 009 (Table 1) as a percentage relative to untreated LPS-stimulated PBMCs. PBMCs treated with microbe growth medium and the anti-inflammatory agent dexamethasone are provided as controls. P-value is determined by ANOVA with Tukey's multiple comparisons. ns: not significant.

FIG. 2B is a dot plot showing the level of TNFα in LPS-stimulated human PBMCs treated with a solution comprising 5% supernatant from microbe 009 in PBMC cell medium (Table 1) relative to untreated LPS-stimulated PBMCs. PBMCs treated with a vehicle control (PYG; 5% solution) and dexamethasone are provided as controls.

FIG. 2C is a bar graph showing the level of TNFα (pg/mL) in LPS-stimulated human PBMCs treated with a solution comprising 2.5%, 5%, or 10% supernatant from microbe 009 in PBMC cell medium (400 μL total volume) (Table 1). Untreated LPS-stimulated PBMCs are shown as a control. Asterisks indicate significant differences, as assessed by ANOVA with Tukey's multiple comparisons.

FIG. 3A is a diagram showing an experimental protocol for the treatment of cells with bacterial supernatants. THP-1-DUAL™ cells are incubated for two hours with the <3 KDa fraction of a microbial supernatant (pretreatment), challenged with either TNFα or LPS for four hours, and assessed for NFκB activity by measurement of a reporter protein (secreted embryonic alkaline phosphatase) using QUANTI-Blue™ solution (InvivoGen).

FIG. 3B is a bar graph showing the level of NFκB activity in LPS-stimulated THP-1-DUAL™ cells treated with a supernatant from microbe 001, 004, 005, or 009 (Table 1). Untreated THP-1-DUAL™ cells, a media control, LPS-stimulated THP-1-DUAL™ cells, and THP-1-DUAL™ cells treated with iIKK (IKK complex inhibitor) are shown as controls. Percentages show percent reduction relative to the LPS-stimulated control. P-value is determined by ANOVA with Tukey's multiple comparisons.

FIG. 3C is a bar graph showing the level of NFκB activity in TNFα-stimulated and LPS-stimulated THP-1-DUAL™ cells treated with a supernatant from microbe 009 (Table 1). Untreated THP-1-DUAL™ cells, TNFα-stimulated THP-1-DUAL™ cells, and LPS-stimulated THP-1-DUAL™ cells are shown as controls. P-value is determined by ANOVA with Tukey's multiple comparisons.

FIG. 4A is a diagram showing an experimental protocol for the treatment of cells with bacterial supernatants. THP-1-DUAL™ cells are challenged for two hours with either TNFα or LPS, then incubated for 24 hours with the <3 KDa fraction of a microbial supernatant. Cells are then assessed for NFκB activity.

FIG. 4B is a bar graph showing the level of NFκB activity in TNFα-stimulated and LPS-stimulated THP-1-DUAL™ cells treated with a supernatant from microbe 016 (Table 1). Untreated THP-1-DUAL™ cells, TNFα-stimulated THP-1-DUAL™ cells, and LPS-stimulated THP-1-DUAL™ cells are shown as controls. P-value is determined by ANOVA with Tukey's multiple comparisons.

FIG. 4C is a bar graph showing the level of NFκB activity in TNFα-stimulated THP-1-DUAL™ cells treated with a supernatant from microbe 001, 003, 004, 005, 006, 007, 009, 010, 011, 012, 013, 014, 015, 016, 017, 018, 019, 021, 023, 025, or 030 (Tables 1 and 2). Untreated THP-1-DUAL™ cells, a media control (BHI), and TNFα-stimulated THP-1-DUAL™ cells are shown as controls. Asterisks indicate significant differences, as assessed by ANOVA with Tukey's multiple comparisons.

FIG. 4D is a bar graph showing the level of NFκB activity in LPS-stimulated THP-1-DUAL™ cells treated with a supernatant from microbe 001, 003, 004, 005, 006, 007, 009, 010, 011, 012, 013, 014, 015, 016, 017, 018, 019, 021, 023, 025, or 030 (Tables 1 and 2). Untreated THP-1-DUAL™ cells, a media control (BHI), and LPS-stimulated THP-1-DUAL™ cells are shown as controls. Asterisks indicate significant differences, as assessed by ANOVA with Tukey's multiple comparisons.

FIG. 5A is a diagram showing an experimental protocol for the treatment of cells with bacterial supernatants. PBMCs are seeded in tissue culture well plates for 24 hours and are pre-treated for 2 hours with 5% v/v of the <3 KDa fraction of a microbial supernatant (microbe-005 or microbe-009; Table 1), stimulated with 100 ng/mL LPS, and incubated for 6 to 24 hours. Unused bacterial media is used as a control. Samples are then assessed for cytokine responses using a 48HD multi-plex LUMINEX® assay.

FIG. 5B is a set of bar graphs showing inflammatory cytokine levels (pg/mL) in LPS-challenged PBMCs pre-treated with the <3 KDa fraction of microbe-009 as shown in FIG. 5A. PBMCs treated with unused bacterial media are shown as a control. Data represent the mean±SD of cytokine concentration in a triplicate set for each condition.

FIG. 5C is a set of bar graphs showing inflammatory cytokine levels (pg/mL) in LPS-challenged PBMCs pre-treated with the <3 KDa fraction of microbe-005 as shown in FIG. 5A. PBMCs treated with unused bacterial media are shown as a control. Data represent the mean±SD of cytokine concentration in a triplicate set for each condition.

### DETAILED DESCRIPTION OF THE INVENTION

### Definitions

The term “isolated microorganism,” as used herein, refers to a microorganism (e.g., a unicellular microorganism (e.g., a bacterium)) that is removed or purified from its natural environment. An isolated microorganism may be obtained by any method or combination of methods, e.g., through the use of cell cultures.

The term “modulating,” as used herein, refers to an observable change in the level of a marker in a subject, as measured using techniques and methods known in the art for the measurement of the marker. Modulating the marker level in a subject may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, at least 100%, or more than 100% or more relative to prior to administration; e.g., up to 100% relative to prior to administration). In some embodiments, modulating is increasing the level of a marker in a subject. Increasing the marker level in a subject may result in an increase of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, 100%, or more than 100% more relative to prior to administration). In other embodiments, modulating is decreasing the level of a marker in a subject. Decreasing the marker level in a subject may result in a decrease of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95%, at least 98%, or 100% or more relative to prior to administration; e.g., up to 100% relative to prior to administration). In embodiments in which a parameter is increased or decreased (or reduced) in a subject following a step of administering a composition described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10, or more administrations, e.g., as part of a dosing regimen for the subject).

The term “pharmaceutical composition,” as used herein, represents a composition formulated with a pharmaceutically acceptable excipient, and for example manufactured or sold with the approval of a governmental regulatory agency as part of a therapeutic regimen for the treatment of a disease, disorder, or condition in a mammal. In some examples, the pharmaceutical composition is a pre-approved composition.

The term “pharmaceutical dosage form,” as used herein, represents those pharmaceutical compositions intended for administration to a subject as is, without further modification (e.g., without reconstitution).

The term “physiologically acceptable composition,” as used herein, represents a composition that, within the scope of sound medical judgment, is suitable for use in contact with the tissues of humans and animals without undue toxicity, irritation, allergic response and the like and are commensurate with a reasonable benefit/risk ratio. A physiologically acceptable composition may be a comestible composition (e.g., a food product or a nutraceutical) or a pharmaceutical composition. A nutraceutical composition may be a nutraceutical oral formulation (e.g., a tablet, powder, lozenge, sachet, cachet, elixir, suspension, emulsion, solution, syrup, or soft or hard gelatin capsule), food additive (e.g., a food additive as defined in 21 C.F.R. § 170.3), food product (e.g., food for special dietary use as defined in 21 C.F.R. § 105.3), or dietary supplement (e.g., where the active agent is a dietary ingredient (e.g., as defined in 21 U.S.C. § 321(ff))).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject.

The term “supernatant,” as used herein, refers to (i) the liquid portion of a composition including a live microorganism or (ii) dry non-aqueous component(s) of the liquid portion (e.g., dry components obtained or isolated from the liquid portion) of a composition including a live microorganism. A supernatant includes microbial metabolites produced by the live organism. A supernatant can include medium in which a live microorganism is cultured.

“Treatment” and “treating,” as used herein, refer to the medical management of a subject with the intent to improve, ameliorate, stabilize, prevent, or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “condition modulated by an inflammatory pathway,” as used herein, refers to a condition that is characterized by dysregulation of an inflammatory pathway and/or that may be ameliorated by a change in a level of activity of an inflammatory pathway. The inflammatory pathway may be, e.g., a NFκB pathway (e.g., an LPS-driven NFκB pathway or a TNFα-driven NFκB pathway) or a TNFα pathway. In some examples, the condition is an inflammatory condition, e.g., an inflammatory bowel disease (IBD), an allergy, or an autoimmune disease. Specific examples include asthma, rheumatoid arthritis, dermatitis (atopic or contact), psoriasis, psoriatic arthritis, multiple sclerosis, celiac disease, glomerulonephritis, hepatitis, reperfusion injury, and transplant rejection. In some examples, the condition modulated by an inflammatory pathway is an IBD.

The term “inflammation marker,” as used herein, refers to any marker (e.g., a DNA, RNA, polypeptide, protein, small molecule, or other molecule) that can be used as an indicator of inflammation, e.g., as an indicator of activity or lack of activity in an individual, a tissue, an organ, or a sample from an individual. The level of the inflammation marker may be increased or decreased in a sample from a patient having an inflammatory condition relative to a healthy patient. The inflammation marker may be associated with an inflammatory pathway, e.g., a NFκB pathway (e.g., an LPS-driven NFκB pathway or a TNFα-driven NFκB pathway) or a TNFα pathway. In some examples, the inflammation marker is NFκB, TNFα, TGFβ, IFNγ, MCP-1, CCL20, IL12p40, IL1β, IL-2, IL-4, IL-5, IL-6, IL-8, IL-10, IL-13, IL-17 or a combination thereof; IL1β, IL8, IL17A, or a combination thereof; MCP-1, CCL20, or a combination thereof; M2-PK pyruvate kinase, osteoprotegerin, MPO, HMGB1, CHI3L1, HBD2, MMP, calprotectin, lactoferrin, pANCA, ASCA, or a combination thereof; or SAA, eotaxin-1, or a combination thereof.

The term “inflammatory bowel disease” or “IBD,” as used herein, refers to a condition of the bowel, e.g., the small intestine, large intestine, mouth, esophagus, stomach, rectum, and/or anus, that is characterized by inflammation. In some examples, the IBD is ulcerative colitis (UC) or Crohn's disease. In other examples, the IBD is microscopic colitis (e.g., collagenous colitis or lyphocytic colitis), diversion colitis, Behcet's disease, or indeterminate colitis.

Microorganisms, Lysates Thereof, and Supernatants Therefrom

Microorganisms

In some aspects, the invention provides a composition comprising an isolated microorganism (e.g., an isolated bacterium) of Table 1.

TABLE 1
Microorganisms

| | Representative | |
|---|---|---|
| Microorganism | strain | Microbe ID |
| Acidaminococcus intestini | DSM 21505 | microbe-064 |
| Acidaminococcus sp. HPA0509 | HM-853 | microbe-065 |
| Akkermansia muciniphila | DSM 22959 | microbe-071 |
| Alistipes putredinis | DSM 17216 | microbe-037 |
| Alistipes finegoldii | DSM 17242 | microbe-016 |
| Anaerostipes hadrus | DSM 3319 | microbe-069 |
| Bacteroides dorei | DSM 17855 | microbe-005 |
| Bacteroides salanitronis | DSM 18170 | microbe-028 |
| Bacteroides stercoris | ATCC 43183 | microbe-017 |
| Bacteroides cellulosilyticus | HM-726 | microbe-066 |
| CL02T12C19 | | |
| Bacteroides cellulosilyticus | DSM 14838 | microbe-006 |
| Bacteroides dorei 5 1 36/D4 | HM-29 | microbe-058 |
| Bacteroides dorei CL02T00C15 | HM-717 | microbe-060 |
| Bacteroides dorei CL02T12C06 | HM-719 | microbe-059 |
| Bacteroides dorei CL03T12C01 | HM-718 | microbe-061 |
| Bacteroides fragilis CL03T12C07 | HM-714 | microbe-048 |
| Bacteroides massiliensis | DSM 17679 | microbe-067 |
| Bacteroides salyersiae | ATCC BAA-997 | microbe-050 |
| Bacteroides sp. 4 1 36 | HM-258 | microbe-046 |
| Bacteroides sp. 9 1 42FAA | HM-27 | microbe-062 |
| Bacteroides sp. D20 | HM-189 | microbe-047 |
| Bacteroides uniformis | ATCC 8492 | microbe-051 |
| Bacteroides vulgatus | ATCC 8482 | microbe-007 |
| Bacteroides vulgatus CL09T03C04 | HM-720 | microbe-057 |
| Clostridium scindens | ATCC 35704 | microbe-063 |
| Clostridium leptum | DSM 753 | microbe-033 |
| Clostridium orbiscindens 1 3 50AFAA | HM-303 | microbe-044 |
| Coprococcus comes | ATCC 27758 | microbe-039 |
| Eubacterium rectale | DSM 17629 | microbe-031 |
| Eubacterium ventriosum | ATCC 27560 | microbe-070 |
| Faecalibacterium prausnitzii KLE1255 | HM-473 | microbe-072 |
| Faecalibacterium prausnitzii A2-165 | DSM 17677 | microbe-038 |
| Flavonifractor plautii | ATCC 29863 | microbe-049 |
| Flavonifractor plautii | DSM 6740 | microbe-053 |
| Lachnospiraceae bacterium 7 1 58FAA | HM-153 | microbe-045 |
| Odoribacter splanchnicus | DSM 20712 | microbe-023 |
| Oscillibacter sp. KLE 1728 | HM-1030 | microbe-042 |
| Parabacteroides distasonis | ATCC 8503 | microbe-009 |
| Parabacteroides merdae CL03T12C32 | HM-730 | microbe-056 |
| Parabacteroides sp. D13 | HM-77 | microbe-041 |
| Parabacteroides merdae | ATCC 43184 | microbe-030 |
| Parabacteroides merdae CL09T00C40 | HM-729 | microbe-043 |
| Phascolarctobacterium faecium | DSM 14760 | microbe-052 |
| Ruminococcus lactaris | ATCC 29176 | microbe-036 |
| Ruminococcus lactaris CC59 002D | HM-1057 | microbe-040 |
| Bacteroides fragilis | ATCC 25285 | microbe-004 |
| Bacteroides helcogenes | ATCC 35417 | microbe-001 |
| Bacteroides xylanisolvens XB1A | DSM 18836 | microbe-014 |

DSM: German Collection of Microorganisms and Cell Cultures GmbH
ATCC: American Type Culture Collection
HM: BEI Resources

TABLE 2
Additional microorganisms

| Microorganism | Microbe ID |
|---|---|
| Bacteroides caccae ATCC 43185 | microbe-003 |
| Eubacterium rectale ATCC 33656 | microbe-010 |
| Bacteroides ovatus ATCC 8483 | microbe-011 |
| Bacteroides caecimuris DSM 26085 | microbe-012 |
| Alistipes shahii ATCC BAA-1179 | microbe-013 |
| Bacteroides thetaiotaomicron ATCC 29148 | microbe-015 |
| Coprobacter fastidiosus DSM 26242 | microbe-019 |
| Flavonifractor plautii ATCC 49531 | microbe-020 |
| Clostridium bolteae ATCC BAA-613 | microbe-021 |
| Bacteroides heparinolyticus DSM 23917 | microbe-025 |

The microorganism (e.g., bacterium) may be isolated (e.g., isolated from a culture, a sample, or a material) using techniques and methods known in the art. In some examples, the microorganism is obtained from a stock center, e.g., the American Type Culture Collection (ATCC®) or the German Collection of Microorganisms and Cell Cultures GmbH (DSM). In some examples, identifying information such as that provided in the tables herein can be used in obtaining the microorganism(s). In other examples, the microorganism is obtained from a biological sample. For example, in some instances, the microorganism is a purified population obtained from a microbiotal material such as a fecal material. In such instances, the microorganism can be identified based on features including, e.g., 16S rDNA sequence analysis. The level of sequence identity used in the identification can be, e.g., at least 95%, 96%, 97%, 98%, or 99% (e.g., 100%) identity to full length, V4, or V6 region 16S rDNA sequences, as is known in the art. Determination of sequence identity can be carried out using standard approaches including, e.g., default values of the alignment programs, for example, BLAST (blast.ncbi.nlm.nih.gov). Levels of identity as described herein can also be used to identify additional, related microorganisms that can be used in the invention. In some examples, a bacterial population that is directly isolated from a fecal material does not result from any culturing or other process that results in or is intended to result in replication of the population after obtaining the fecal material.

In some instances, the microorganism is isolated from a live bacterial culture. The live cultured bacteria may be prepared using cell culture techniques and methods known in the art. Typically, isolation of live cultured bacterial cells includes separation from the broth, e.g., by centrifuging, filtration, or decanting. The cells separated from the fermentation broth are optionally washed by water, saline (e.g., 0.9% NaCl), or with any suitable buffer. In some instances, the wet cell mass obtained is dried. The cell mass may be dried by any suitable method, e.g., dried by lyophilization.

The microorganism in the composition may be, e.g., a live microorganism (e.g., a live bacterium, e.g., a live cultured bacterium) a dead microorganism, (e.g., a killed microorganism, e.g., a heat-killed microorganism or an irradiated microorganism), a microorganism (e.g., a bacterium) in a vegetative state, a microorganism (e.g., a bacterium) in a spore form (e.g., an endospore), or a combination thereof.

In some examples, the composition comprises a single isolated microorganism, i.e., comprises one microorganism of Table 1. In other examples, the composition comprises a combination of microorganisms, e.g., comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or all 48 microorganisms of Table 1, e.g., comprises 2-5, 5-10, 10-15, 15-20, 20-25, 25-30, 30-35, 35-40, 40-45, or 45-48 of these microorganisms. In some examples, the composition comprises all of the microorganisms of Table 1.

In some examples, the preparation is substantially free of contaminating bacteria (i.e., bacteria other than the microorganism or microorganisms intended to be isolated from the culture, sample, or material). In some aspects, the preparation is 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% free of contaminating bacteria.

The composition comprising the microorganism may be further processed, e.g., sterilized and/or processed to remove endotoxin.

In some embodiments, the isolated microorganism has been grown in a culture medium selected from the group consisting of brain heart infusion (BHI) media, BHI-HK media (BHI media supplemented with Vitamin K₁ and Hemin), and Brucella blood agar (BRU-AS) media. In some embodiments, (a) the isolated microorganism is microbe-005, microbe-056, or microbe-059 (Table 1) and the culture medium was BHI media; (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 (Table 1) and the culture medium was BHI-HK media; or (c) the isolated microorganism is microbe-041, microbe-043, microbe-045, or microbe-050 (Table 1) and the culture medium was BRU-AS media.

In some aspects, the invention provides a method of growing an isolated microorganism of Table 1, the method comprising culturing the isolated microorganism in a culture medium selected from the group consisting of BHI media, BHI-HK media, and BRU-AS media.

In some embodiments, (a) the isolated microorganism is microbe-005, microbe-056, or microbe-059 and the culture medium is BHI media; (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium is BHI-HK media; or (c) the isolated microorganism is microbe-041, microbe-043, microbe-045, or microbe-050 and the culture medium is BRU-AS media.

Microbial Lysates

In some aspects, the invention provides a composition comprising a lysate of an isolated microorganism (e.g., an isolated bacterium) of Table 1, e.g., a lysate of a bacterium described in Section IA herein. The microorganisms may be lysed prior to or after their incorporation into the composition (e.g., physiologically acceptable composition). Lysis of microorganisms (e.g., bacteria) may be performed using techniques and methods known in the art for cell lysis. In one exemplary method for preparing a microbial lysate, spun-down cells are suspended in 20 mL MeOH and sonicated for 20 minutes. Cell debris is then spun down, and collected supernatant is dried under vacuum using a rotary evaporator. The dried extract is resuspended in MeOH/H₂O(1:1) and filtered to remove components having a molecular weight of more than 3 kDa.

In some examples, the preparation comprising the lysed microorganism is substantially free of non-lysed bacteria, e.g., 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% free of non-lysed bacteria.

In some examples, the composition comprises a lysate of a single isolated microorganism, i.e., comprises a lysate of one microorganism of Table 1. In other examples, the composition comprises lysates of a combination of microorganisms, e.g., comprises lysates of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or all 48 microorganisms of Table 1, e.g., comprises lysates of 2-5, 5-10, 10-15, 15-20, 20-25, 25-30, 30-35, 35-40, 40-45, or 45-48 microorganisms. In some examples, the composition comprises lysates of all of the microorganisms of Table 1.

The composition comprising the lysate of the isolated microorganism may be further processed, e.g., sterilized and/or processed to remove endotoxin.

In some examples, the composition comprising the lysate is processed to remove compounds having a particular molecular weight, e.g., is processed to remove compounds having a molecular weight above or below a threshold. The processing may comprise, e.g., filtration.

For example, in some instances, the composition comprising the lysate of the isolated microorganism is processed (e.g., filtered) to remove components having a molecular weight of more than 100 kDa, more than 50 kDa, more than 25 kDa, more than 20 kDa, more than 15 kDa, more than 10 kDa, more than 9 kDa, more than 8 kDa, more than 7 kDa, more than 6 kDa, more than 5 kDa, more than 4 kDa, more than 3.5 kDa, more than 3 kDa, more than 2.5 kDa, more than 2 kDa, more than 1.5 kDa, more than 1 kDa, more than 0.5 kDa, or more than 0.25 kDa. In some examples, the composition has been processed to remove all components having a molecular weight of more than 3 kDa.

In some instances, the composition comprising the lysate of the isolated microorganism is processed (e.g., filtered) to remove components having a molecular weight of less than 100 kDa, less than 50 kDa, less than 25 kDa, less than 20 kDa, less than 15 kDa, less than 10 kDa, less than 9 kDa, less than 8 kDa, less than 7 kDa, less than 6 kDa, less than 5 kDa, less than 4 kDa, less than 3.5 kDa, less than 3 kDa, less than 2.5 kDa, less than 2 kDa, less than 1.5 kDa, less than 1 kDa, less than 0.5 kDa, or less than 0.25 kDa. In some examples, the composition has been processed to remove all components having a molecular weight of less than 3 kDa.

Microbial Supernatants

In some aspects, the invention provides a composition comprising a supernatant of a culture of an isolated microorganism (e.g., an isolated bacterium) of Table 1, e.g., a supernatant of a culture of a bacterium described in Section IA herein, e.g., comprises a liquid medium in which the microorganism has been cultured or a fraction thereof. The supernatant may contain molecules secreted or otherwise produced by the bacteria, e.g., may contain polypeptides, lipids, nucleic acids, and/or small molecules secreted or otherwise produced by the bacteria. The bacterium may be cultured and the supernatant may be produced using methods known in the art. For example, the supernatant may be produced by centrifuging the culture and separating the supernatant from pelleted microorganism. In other examples, the supernatant is produced by filtering the culture medium to remove bacteria.

In some examples, the supernatant is substantially free of bacteria, e.g., 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% free of bacteria (e.g., live bacteria).

In some examples, the composition comprises a supernatant from a single isolated microorganism, i.e., comprises a supernatant from one microorganism of Table 1. In other examples, the composition comprises supernatants from a combination of microorganisms, e.g., comprises supernatants from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or all 48 microorganisms of Table 1, e.g., comprises supernatants from 2-5, 5-10, 10-15, 15-20, 20-25, 25-30, 30-35, 35-40, 40-45, or 45-48 microorganisms. In some examples, the composition comprises supernatants from all of the microorganisms of Table 1.

The supernatant from the isolated microorganism may be further processed, e.g., sterilized and/or processed to remove endotoxin.

In some examples, the supernatant is processed to remove compounds having a particular molecular weight, e.g., is processed to remove compounds having a molecular weight above or below a threshold. The processing may comprise, e.g., filtration.

For example, in some instances, the supernatant is processed (e.g., filtered) to remove components having a molecular weight of more than 100 kDa, more than 50 kDa, more than 25 kDa, more than 20 kDa, more than 15 kDa, more than 10 kDa, more than 9 kDa, more than 8 kDa, more than 7 kDa, more than 6 kDa, more than 5 kDa, more than 4 kDa, more than 3.5 kDa, more than 3 kDa, more than 2.5 kDa, more than 2 kDa, more than 1.5 kDa, more than 1 kDa, more than 0.5 kDa, or more than 0.25 kDa. In some examples, the supernatant has been processed to remove all components having a molecular weight of more than 3 kDa.

In some instances, the supernatant is processed (e.g., filtered) to remove components having a molecular weight of less than 100 kDa, less than 50 kDa, less than 25 kDa, less than 20 kDa, less than 15 kDa, less than 10 kDa, less than 9 kDa, less than 8 kDa, less than 7 kDa, less than 6 kDa, less than 5 kDa, less than 4 kDa, less than 3.5 kDa, less than 3 kDa, less than 2.5 kDa, less than 2 kDa, less than 1.5 kDa, less than 1 kDa, less than 0.5 kDa, or less than 0.25 kDa. In some examples, the supernatant has been processed to remove all components having a molecular weight of less than 3 kDa.

Combinations of Microorganisms, Supernatants, and Lysates

In some instances, the composition comprises two or all three of a microorganism, a lysate of a microorganism, and a supernatant of a culture of an isolated microorganism of Table 1. In some instances, the composition comprises two or all three of a microorganism, a lysate of the microorganism, and a supernatant from the microorganism. In other instances, the composition comprises two or all three of a microorganism, a lysate, and a supernatant, wherein the microorganism in the composition, the microorganism from which the lysate is derived, and the microorganism from which the supernatant is derived are different microorganisms.

### Formulations and Compositions

Compositions (e.g., physiologically acceptable compositions) described herein may be prepared using techniques and methods known in the art.

The active agents (e.g., microorganisms, lysates thereof, and supernatants therefrom) disclosed herein may be formulated into physiologically acceptable compositions (e.g., pharmaceutical or nutraceutical compositions) for administration to human subjects in a biologically compatible form suitable for administration in vivo. Pharmaceutical and nutraceutical compositions typically include an active agent as described herein and a physiologically acceptable excipient (e.g., a pharmaceutically acceptable excipient). The active agents useful in physiologically acceptable compositions described herein include isolated microorganisms, lysates thereof, and supernatants therefrom.

The active agents (e.g., microorganisms, lysates thereof, and supernatants therefrom) described herein may be administered, for example, by oral, rectal (e.g., by enema, ointment, or suppository), enteral, parenteral, buccal, sublingual, nasal, patch, pump, or transdermal administration, and the pharmaceutical or nutraceutical compositions formulated accordingly. Parenteral administration includes intravenous, intraperitoneal, subcutaneous, intramuscular, transepithelial, nasal, intrapulmonary, intrathecal, and topical modes of administration. Parenteral administration may be by continuous infusion over a selected period of time.

For human use, an active agent disclosed herein can be administered alone or in admixture with a pharmaceutical or nutraceutical carrier selected regarding the intended route of administration and standard pharmaceutical practice. Pharmaceutical and nutraceutical compositions for use in accordance with the present invention thus can be formulated in a conventional manner using one or more physiologically acceptable carriers including excipients and auxiliaries that facilitate processing of active agents disclosed herein into preparations which can be used pharmaceutically.

This disclosure also includes pharmaceutical and nutraceutical compositions which can contain one or more physiologically acceptable carriers. In making the pharmaceutical or nutraceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of, e.g., tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions may include additional agents, e.g., preservatives. Nutraceutical compositions may be administered enterally (e.g., orally). A nutraceutical composition may be a nutraceutical oral formulation (e.g., a tablet, powder, lozenge, sachet, cachet, elixir, suspension, emulsion, solution, syrup, or soft or hard gelatin capsule), a food additive (e.g., a food additive as defined in 21 C.F.R. § 170.3), a food product (e.g., food for special dietary use as defined in 21 C.F.R. § 105.3), or a dietary supplement (e.g., where the active agent is a dietary ingredient (e.g., as defined in 21 U.S.C. § 321(ff))). Active agents (e.g., microorganisms, lysates thereof, and supernatants therefrom) can be used in nutraceutical applications and as food additive or food products. Non-limiting examples of compositions including an active agent of the invention are a bar, drink, shake, powder, additive, gel, or chew.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical and nutraceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical and nutraceutical formulation. In preparing a formulation, the active agents can be milled to provide the appropriate particle size prior to combining with the other ingredients. If the active agent is substantially insoluble, it can be milled to a particle size of less than 200 mesh. If the active agent is substantially water soluble, the particle size can be adjusted by milling to provide a substantially uniform distribution in the formulation, e.g., about 40 mesh.

In instances in which the composition comprises a microorganism (e.g., a bacterium), the physiologically acceptable compositions described herein may be formulated such that a single unit dose contains at least about 1×10⁴ colony forming units (cfu) of the bacteria, and a single consumed unit may contain, e.g., about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ cfu of the bacteria; or contain at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ cfu. The concentration of bacteria of a given species is e.g., 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ viable bacteria per gram of the physiologically acceptable composition. The bacteria are administered, for example, as live cultured bacteria, in vegetative or spore form or as a combination of vegetative and spore forms. Alternatively, the bacteria are provided as purified populations obtained from a microbiotal material such as a fecal material.

The amount of bacteria contained in a unit dose of a physiologically acceptable composition may be about 0.001 mg to about 1 mg, about 0.5 mg to about 5 mg, about 1 mg to about 1000 mg, about 2 mg to about 200 mg, about 2 mg to about 100 mg, about 2 mg to about 50 mg, about 4 mg to about 25 mg, about 5 mg to about 20 mg, about 10 mg to about 15 mg, about 50 mg to about 200 mg, about 200 mg to about 1000 mg, or about 1, 2, 3, 4, 5 or more than 5 g per dose or composition; or 0.001 mg to 1 mg, 0.5 mg to 5 mg, 1 mg to 1000 mg, 2 mg to 200 mg, or 2 mg to 100 mg, or 2 mg to 50 mg, or 4 mg to 25 mg, or 5 mg to 20 mg, or 10 mg to 15 mg, or 50 mg to 200 mg, or 200 mg to 1000 mg, or 1, 2, 3, 4, 5 or more than 5 g per dose or composition.

A physiologically acceptable composition described herein may be a food product, e.g., a medical food product. A food product may be, e.g., a dairy product, an infant food product, a fruit-containing food product, a vegetable-containing food product, or a sports nutrition food product. The food products can be applied in infant diets, adult diets and special diets, including diets requiring medical foods or functional foods. The bacterial compositions can be incorporated in powder or in liquid form in foods used by the general population, particularly milk and milk-derived products, especially fermented milk and cheeses; cereals and derivatives, including bread, bread doughs, cakes, cookies, crackers, extruded snacks; soups and other similar products in dehydrated form; fermented meat products; fruit and vegetable derivatives, juices and soft drinks; foods for specific nutritional uses, including infant milk, infant cereals, ready-to-eat infant foods, etc. They can also be found in food supplements and special formulas for oral and enteral nutrition for clinical use. In other embodiments, the foodstuff is yogurt, kefir, yakult, miso, natto, tempeh, kimchee, sauerkraut, water, coffee, tea, beer, wine, liquor, alcoholic mixed drinks, soups, frozen desserts, fried foods, pasta products, potato products, rice products, corn products, wheat products, dairy products, confectioneries, hard candies, nutritional bars, and breakfast cereals. Generally, the bacterial population includes bacteria present in the foodstuff in an amount from about 10⁴ to about 10¹² cfu per gram of foodstuff, e.g., from 10⁴ to 10¹² cfu per gram of foodstuff. Additionally, the bacterial population may be stabilized to prevent spoilage of the foodstuff. Generally, the bacterial population is present in the liquid in an amount from about 10⁴ to about 10¹² cfu per gram of liquid, e.g., from 10⁴ to 10¹² cfu per gram of liquid. The beverage may be hot, warm, room temperature, cool, or cold.

Physiologically acceptable compositions described herein may be prepared using methods known in the art and those described herein. Typically, preparation of a physiologically acceptable composition involves combining an effective amount of the active agent (e.g., a microorganism composition and/or microbial metabolite) with a carrier. For comestible compositions, e.g., orally administered pharmaceutical composition and food products (foodstuffs and beverages), an effective amount of the bacterial population is a population containing an amount of bacteria such that the population is at least partially retained in the gastrointestinal tract of a subject that consumes the composition. For example, the bacterial population contains bacteria present in the carrier in an amount from about 10⁴ to about 10¹² cfu per gram of carrier, e.g., from 10⁴ to 10¹² cfu per gram of carrier.

A comestible composition may be provided in a unit dose or serving of 5-500 g, e.g., 5-15 g, 15-50 g, 25-75 g, 50-100 g, 100-200 g, 200-300 g, 300-400 g, or 400-500 g. For example, a yogurt composition can be about 4, 6, 8, 10 or 12 ounces, or a quarter, half, three-quarters or whole cup. In one embodiment, a typical serving size for a beverage product such as a fluid is about 10-500 ml, e.g., 10-25 ml, 25-50 ml, 50-75 ml, 75-100 ml, 100-150 ml, 150-200 ml, 250-300 ml, 300-400 ml, or 400-500 ml.

A physiologically acceptable composition may be a pharmaceutical composition, e.g., for administration in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; or liquid forms disclosed in U.S. Pat. Nos. 4,625,494, 4,478,822, and 5,610,184; each of which is incorporated herein by reference in its entirety. Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in stomach or in parts of the gut (e.g., colon, lower intestine) other than the stomach. All formulations for oral administration can be in dosages suitable for such administration. The push-fit capsules can contain the active ingredients in admixture with filler, such as lactose, binders such as starches, and/or lubricants such as talc or magnesium stearate and, optionally, stabilizers. In soft capsules, the active compounds (prebiotics or probiotics) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or dragee coatings for identification or to characterize different combinations of active compound doses. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil. Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

A physiologically acceptable composition may be a softgel formulation. A softgel can contain a gelatin-based shell that surrounds a liquid fill. The shell can be made of gelatin, plasticizer (e.g., glycerin and/or sorbitol), modifier, water, color, antioxidant, or flavor. The shell can be made with starch or carrageenan. The outer layer can be enteric coated. In one embodiment, a softgel formulation can include a water or oil soluble fill solution, or suspension of a composition, for example, a prebiotic composition, covered by a layer of gelatin.

An enteric coating can control the location of where a physiologically acceptable composition is absorbed in the digestive system. For example, an enteric coating can be designed such that a physiologically acceptable composition does not dissolve in the stomach but rather travels to the small intestine, where it dissolves. An enteric coating can be stable at low pH (e.g., in the stomach) and can dissolve at higher pH (e.g., in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac, cellulose acetate phthalate, polyvinylacetate phthalate, and methacrylic acid. Enteric coatings can be used to (1) prevent the gastric juice from reacting with or destroying the active substance, (2) prevent dilution of the active substance before it reaches the intestine, (3) ensure that the active substance is not released until after the preparation has passed the stomach, and (4) prevent live bacteria contained in the preparation from being killed because of the low pH-value in the stomach. In one embodiment a bacterial composition or the bacterial component of a food or beverage is provided as a tablet, capsule, or caplet with an enteric coating. In one embodiment the enteric coating is designed to hold the tablet, capsule, or caplet together when in the stomach. The enteric coating is designed to hold together in acid conditions of the stomach and break down in non-acid conditions and therefore release the drug in the intestines. Softgel delivery systems can also incorporate phospholipids or polymers or natural gums to entrap a composition, for example, a prebiotic composition, in the gelatin layer with an outer coating to give desired delayed/control release effects, such as an enteric coating.

A physiologically acceptable composition may be provided in a dosage form which includes an effective amount of an active agent (e.g., an isolated microorganism population or a microbial metabolite) and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In one embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule. In another embodiment, a composition including an active agent is provided in effervescent dosage forms. The compositions can also include non-release controlling excipients.

A physiologically acceptable composition (e.g., a physiologically acceptable composition formulated for oral administration) may be provided in the form of enteric-coated pellets, enteric-coated tablet, enteric-coated dragee, or enteric-coated capsules. The compositions can further include glyceryl monostearate 40-50, hydroxypropyl cellulose, hypromellose, magnesium stearate, methacrylic acid copolymer type C, polysorbate 80, sugar spheres, talc, and triethyl citrate. In one embodiment a composition including a bacterial population is provided in the form of enteric-coated granules, for oral administration. The compositions can further include carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

Physiologically acceptable compositions can be formulated in various dosage forms for oral administration. The compositions can also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to known methods and techniques (see, Remington: The Science and Practice of Pharmacy, 21st Ed., Gennaro, Ed., Lippincott Williams & Wilkins (2005); Modified-Release Drug Delivery Technology, Rathbone et al., Eds., Drugs and the Pharmaceutical Science, Marcel Dekker, Inc.: New York, N.Y., 2002; Vol. 126, which are incorporated herein by reference in their entirety). In one embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition including a prebiotic. Furthermore, dosage forms can include acceptable carriers or salts known in the art, such as those described in the Handbook of Pharmaceutical Excipients, American Pharmaceutical Association (1986), incorporated by reference herein in its entirety.

Physiologically acceptable compositions described herein can be in liquid form. The liquid formulations can include, for example, an agent in water-in-solution and/or suspension form; and a vehicle including polyethoxylated castor oil, alcohol, and/or a polyoxyethylated sorbitan mono-oleate with or without flavoring. Each dosage form includes an effective amount of an active agent and can optionally include pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for oral administration. Examples of such vehicles and additives can be found in Remington: The Science and Practice of Pharmacy, 21st Ed., Gennaro, Ed., Lippincott Williams & Wilkins (2005).

Methods of Treatment

Methods of Treating or Preventing Inflammatory Diseases or Conditions

In some aspects, the invention provides a method of treating or preventing a disease or condition in a subject in need thereof. In some examples, the disease or condition is an inflammatory disease, an allergy, or an autoimmune disease. In some examples, the disease or condition is modulated by an inflammatory pathway. Specific examples of diseases and conditions that can be treated or prevented according to the invention include: an inflammatory bowel disease (IBD) (e.g., ulcerative colitis (UC) or Crohn's disease), asthma, rheumatoid arthritis, dermatitis (atopic or contact), psoriasis, psoriatic arthritis, multiple sclerosis, celiac disease, glomerulonephritis, hepatitis, reperfusion injury, and transplant rejection. The method of the invention comprises administering to a subject in need thereof a therapeutically effective amount of a composition comprising an isolated microorganism of Table 1, a lysate thereof, or a supernatant thereof. Exemplary microorganisms, lysates, and supernatants that may be used in the invention, and compositions and formulations comprising such agents, are described in Sections I and II herein.

The composition may be administered to the subject in a single dose or in multiple doses. When multiple doses are administered, the doses may be separated from one another by, for example, 1-24 hours, 1-7 days, 1-4 weeks, 1 month, two months, six months, or more than six months. In some examples, the composition is administered according to a schedule; in other examples, the composition is administered without a predetermined schedule. It is to be understood that, for any particular subject, specific dosage regimes should be adjusted over time according to the individual need and the professional judgment of the person administering or supervising the administration of the compositions.

In examples of the method comprising multiple doses, the duration of treatment may vary. For example, in some embodiments, doses of the composition comprising the microorganism, lysate thereof, or supernatant of a culture thereof are administered to a subject over a time period that is 1-7 days (e.g., administered daily or every 2, 3, 4, 5, 6, or 7 days or administered more frequently for a duration of 1-7 days); 1-12 weeks (e.g., administered weekly or every 2, 3, 4, 5, 6, 7, 8, 9, 10, 11, or 12 weeks or administered more frequently for a duration of 2-12 weeks); or 1-3 months (e.g., administered every 1, 2, or 3 months or administered more frequently for a duration of 1-3 months). In other embodiments, the active agents are administered to the subject over a time period that is, for example, 4-11 months (e.g., administered every 4, 5, 6, 7, 8, 9, 10, or 11 months or administered more frequently for a duration of 4-11 months) or 1-30 years (e.g., administered every 1, 2, 3, 4, 5, 10, 15, 20, 25, or 30 years or administered more frequently for a duration of 1-30 years). In yet other embodiments, the active agents disclosed herein are administered to a subject at the onset of symptoms. In any of these embodiments, the amount of the active agent that is administered may vary during the time period of administration. When an active agent is administered daily, administration may occur, for example, 1, 2, 3, or 4 times per day.

In embodiments in which a parameter is increased or decreased (or reduced) in a subject following a step of administering a composition described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10 or more administrations, e.g., as part of a dosing regimen for the subject).

In some embodiments of methods in which the composition comprises one or more isolated microorganisms, the one or more isolated microorganisms may colonize the gut of a subject. Colonization of the gut by a microorganism may be determined by detecting the microorganism in a sample from the gut (e.g., by detecting one or more nucleotide sequences that may be used to identify the microorganism) of the subject following the administration of the composition comprising the microorganism, e.g., detecting the microorganism in a sample from the gut of the subject at least 1 day, at least 5 days, at least 1 week, at least 2 weeks, at least one month, at least two months, or more than two months following administration of the composition comprising the microorganism.

Methods of Treating Ulcerative Colitis

In some examples, the inflammatory disease is an IBD which is ulcerative colitis (UC). In some examples, the method reduces the occurrence or severity of one or more symptoms of UC relative to a subject who has not been treated with the composition, e.g., reduces the occurrence or severity of one or more symptoms of UC by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition. In some examples, the method reduces the occurrence or severity of one or more symptoms of UC relative to a baseline status of the subject before treatment, e.g., reduces the occurrence or severity of one or more symptoms of UC by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline status of the subject before treatment.

In some examples, the method reduces the likelihood that the subject will develop UC relative to a subject who has not been treated with the composition, e.g., reduces the likelihood that the subject will develop UC by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition.

Methods of Treating Crohn's Disease

In some examples, the inflammatory disease is an IBD which is Crohn's disease. In some examples, the method reduces the occurrence or severity of one or more symptoms of Crohn's disease relative to a subject who has not been treated with the composition, e.g., reduces the occurrence or severity of one or more symptoms of Crohn's disease by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition. In some examples, the method reduces the occurrence or severity of one or more symptoms of Crohn's disease relative to a baseline status of the subject before treatment, e.g., reduces the occurrence or severity of one or more symptoms of Crohn's disease by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline status of the subject before treatment.

In some examples, the method reduces the likelihood that the subject will develop Crohn's disease relative to a subject who has not been treated with the composition, e.g., reduces the likelihood that the subject will develop Crohn's disease by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition.

Methods of Treating or Preventing Conditions Modulated by an Inflammatory Pathway

In some aspects, the invention provides a method of treating or preventing a condition modulated by an inflammatory pathway in a subject in need thereof, the method comprising administering to the subject a therapeutically effective amount of a composition comprising an isolated microorganism of Table 1 a lysate thereof, or a supernatant of a culture thereof. Exemplary microorganisms, lysates, and supernatants that may be used in the invention and compositions and formulations comprising such agents are described in Sections I and II herein.

In some examples, the inflammatory pathway is an NFκB pathway (e.g., an LPS-driven NFκB pathway or a TNFα-driven NFκB pathway) or a TNFα pathway.

In some examples, the method reduces the occurrence or severity of one or more symptoms of the condition modulated by the inflammatory pathway relative to a subject who has not been treated with the composition, e.g., reduces the occurrence or severity of one or more symptoms of the condition modulated by the inflammatory pathway by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition. In some examples, the method reduces the occurrence or severity of one or more symptoms of the condition modulated by the inflammatory pathway relative to a baseline status of the subject before treatment, e.g., reduces the occurrence or severity of one or more symptoms of the condition modulated by the inflammatory pathway by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline status of the subject before treatment.

In some examples, the method reduces the likelihood that the subject will develop the condition modulated by the inflammatory pathway relative to a subject who has not been treated with the composition, e.g., reduces the likelihood that the subject will develop the condition modulated by the inflammatory pathway by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a subject who has not been treated with the composition.

In some examples in which the condition modulated by the inflammatory pathway is characterized by increased activation of the inflammatory pathway, the method reduces the activation of the inflammatory pathway by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline level in the treated subject or relative to a subject who has not been treated with the composition. In some examples, activation of the inflammatory pathway is decreased by at least 20%.

In some examples in which the condition modulated by the inflammatory pathway is characterized by decreased activation of the inflammatory pathway, the method increases the activation of the inflammatory pathway by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline level in the treated subject or relative to a subject who has not been treated with the composition. In some examples, activation of the inflammatory pathway is increased by at least 20%.

In some aspects, the condition modulated by the inflammatory pathway is an IBD, e.g., UC or Crohn's disease, or another disease or condition listed herein.

Methods of Modulating Inflammation Markers

In some aspects, the invention provides a method of modulating an inflammation marker in a subject in need thereof, the method comprising administering to the subject a therapeutically effective amount of a composition comprising an isolated microorganism of Table 1, a lysate thereof, or a supernatant of a culture thereof. Exemplary microorganisms, lysates, and supernatants that may be used in the invention and compositions and formulations comprising such agents are described in Sections I and II herein.

In some examples, the inflammation marker is associated with an NFκB pathway or a TNFα pathway. In some examples, the inflammation marker is NFκB, TNFα, TGFβ, IFNγ, IL12p40, IL-2, IL-4, IL-5, IL-6, IL-10, IL-13, or IL-17. Representative cytokines that are inflammation markers, along with associated mouse models and human disease indications, are provided in Table 3.

In some aspects, the subject is suffering from inflammatory bowel disease or is at risk of developing inflammatory bowel disease. Inflammatory bowel disease markers are known in the art, e.g., intestinal lining inflammation, ulcers in the intestinal lining, TNFα serum levels, IL6 serum levels, IL1β serum levels, NFkB levels (e.g., in an intestinal lining tissue), IL-8 levels (e.g., in an intestinal lining tissue), IL-12 levels (e.g., in an intestinal lining tissue), IL-17A levels, M2-PK pyruvate kinase levels (e.g., in feces), osteoprotegerin levels (e.g., in intestinal lining tissue, serum, or feces), myeloperoxidase (MPO) levels, high mobility group box-1 (HMGB1) levels (e.g., in feces), Chitinase 3-like-1 (CHI3L1) levels (e.g., in serum or feces), human beta defensin 2 (HBD2) levels, matrix metalloproteinase levels (e.g., MMP2 or MMP9; e.g., serum or urine levels), calprotectin levels (e.g., in feces), lactoferrin levels, pANCA levels (e.g., in serum; especially, in subjects suffering from ulcerative colitis), ASCA levels (e.g., in serum; especially, in subjects suffering from Crohn's disease), eotaxin-1 levels, MCP-1 levels (e.g., in an intestinal lining tissue), MIP-1 levels (e.g., in an intestinal lining tissue), MIP-2 levels (e.g., in an intestinal lining tissue), RANTES levels (e.g., in an intestinal lining tissue), CXCL-1 levels (e.g., in an intestinal lining tissue), CCL20 levels (e.g., in an intestinal lining tissue), GCSF levels (e.g., in an intestinal lining tissue), GM-CSF levels (e.g., in an intestinal lining tissue), CRP levels, fibrinogen levels, CD3 levels, C4 levels, sialic acid levels, C1 activation enzyme levels, C3a levels, C5 levels, bradykinin levels, plasminogen activator levels, SAA levels, PGE2 levels, D2 levels, F2/LTA4 levels, B4 levels, C4 levels, D4 levels, cationic protein levels, oxygen derived free radicals, nitrogen derived free radicals, vasoactive amine (e.g., histamine or serotonin) levels, integrin/adhesion molecules levels (e.g., a/B integrins, Ig superfamily, selectins, cadherins), erythrocyte sedimentation rate, procalcitonin levels, white blood cell count, immunoglobulin level (e.g., IgA, IgG, or IgM), CD80 levels, CD86 levels, CD69 levels, MHC levels, IFNγ levels (e.g., in an intestinal lining tissue); levels of inhibition of Dectin-1 mediated NFkB signaling, NFAT levels, levels of induction of regulatory T cells (Tregs), levels of TGFβ, levels of IL-10 expression, levels of Th17 induction blockade, levels of IL-23R, or levels of IL-17A/F in the subject. Thus, the methods described herein may be used, e.g., to reduce TNFα serum levels, IL6 serum levels, and/or IL1β serum levels. Additionally or alternatively, the methods described herein may be used, e.g., to reduce NFkB levels (e.g., in an intestinal lining tissue), IL-8 levels (e.g., in an intestinal lining tissue), MCP-1 levels (e.g., in an intestinal lining tissue), CCL20 levels (e.g., in an intestinal lining tissue), or IFNγ levels (e.g., in an intestinal lining tissue) in the subject. Additionally or alternatively, the methods described herein may be used, e.g., to reduce the intestinal lining inflammation and/or ulcers in the intestinal lining, as observed by colonoscopy. Additionally or alternatively, the methods described herein may be used, e.g., to reduce NFkB, MCP-1, CCL20, or IFNγ levels in the subject in the subject. Additionally or alternatively, the methods described herein may be used, e.g., to reduce TNFα, IL1β, IL6, IL8, or IL17A levels in the subject in the subject. Additionally or alternatively, the methods described herein may be used, e.g., to reduce M2-PK pyruvate kinase, osteoprotegerin, MPO, HMGB1, CHI3L1, HBD2, MMP, calprotectin, lactoferrin, pANCA, or ASCA levels in the subject. Additionally or alternatively, the methods described herein may be used, e.g., to reduce SAA or eotaxin-1 levels in the subject in the subject. Additionally or alternatively, the methods described herein may be used, e.g., to reduce levels of Dectin-1 mediated NFkB signaling; reduce NFAT levels; increase induction of regulatory T cells (Tregs), levels of TGFβ, and/or levels of IL-10 expression; or reduce levels of Th17 induction, levels of IL-23R, or levels of IL-17A/F in the subject.

In some examples, the method reduces a level of the inflammation marker by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline level in the treated subject or relative to a subject who has not been treated with the composition. In some examples, the level of the inflammation marker is decreased by at least 20%.

In some examples, the method increases a level of the inflammation marker by at least 1%, 5%, 10%, 15%, 20%, 25%, 30%, 35%, 40%, 45%, 50%, 55%, 60%, 65%, 70%, 75%, 80%, 85%, 90%, 95%, 99%, or 100% relative to a baseline level in the treated subject or relative to a subject who has not been treated with the composition. In some examples, the level of the inflammation marker is increased by at least 20%.

In some examples, the inflammation marker is present in a sample from a subject having or at risk of developing a disease or condition described herein.

TABLE 3
Mouse models of inflammation (quick readout;
cytokines associated with human disease)

| Associated cytokines | Mouse model | Indication |
|---|---|---|
| TNFα, IL12p40 | TNBS colitis | Ulcerative colitis |
| | DSS colitis | Crohn's Disease |
| TNFα, IL12p40 | Collagen-induced arthritis | Rheumatoid arthritis |
| TNFα, IL-2, IFNγ | KLH-DTH (Th1) | Contact dermatitis |
| | | Allergy |
| IL-4, IL-5, IL-10, IL-13 | FITC dermatitis (Th2) | Atopic dermatitis |
| | MC903 dermatitis (Th2) | Food allergy |
| | DNFB dermatitis (Th2) | Asthma |
| IL-17, TNFα, IL12p40 | Imiquimod dermatitis (Th17) | Psoriasis |
| | | Psoriasis arthritis |
| Inhibit IL17, IL-2, TNFα | Experimental autoimmune | Multiple sclerosis |
| Promote IL-10, TGFβ | encephalomyelitis | (autoimmune) |

The following examples are meant to illustrate the invention. They are not meant to limit the invention in any way.

### EXAMPLES

### Example 1. Preparation of Bacterial Supernatants

Bacterial supernatants were prepared the following steps. Microbes were purchased from the American Type Culture Collection (ATCC®) and the Leibniz Institute DSMZ and were anaerobically cultured in BHI (AS-872 Anaerobe Systems), YCFAC (AS-680, Anaerobe Systems), CM (AS-811, Anaerobe Systems), PYG (AS-822, Anaerobe Systems), or RCM (AS-606, Anaerobe Systems) media for 72 hours. Culture supernatants were sterilized using 220 nm filters, and endotoxins were removed using Pierce™ High Capacity Endotoxin Removal Spin Columns (ThermoFisher Scientific) to produce sterilized, endotoxin-removed supernatants. In some cases, supernatants are further fractionated using size exclusion columns to separate molecules that are below 3 KDa or above 3 KDa.

Table 1 provides a list of the strains from which supernatants were prepared.

### Example 2. LPS-Induced NFκB Signaling Assessment

NFκB reporters of human THP-1-DUAL™ cell lines (InvivoGen) were grown in RPMI, and 25,000 cells were seeded per well on a 96 well plate for 20 hours.

THP-1-DUAL™ cells (InvivoGen) were incubated with bacterial supernatants prepared as described in Example 1 (<3 KDa fraction) for 2 hours, followed by LPS challenge for 4 hours. NFkB activity was then assessed by measurement of a reporter protein (secreted embryonic alkaline phosphatase) using QUANTI-Blue™ solution (InvivoGen).

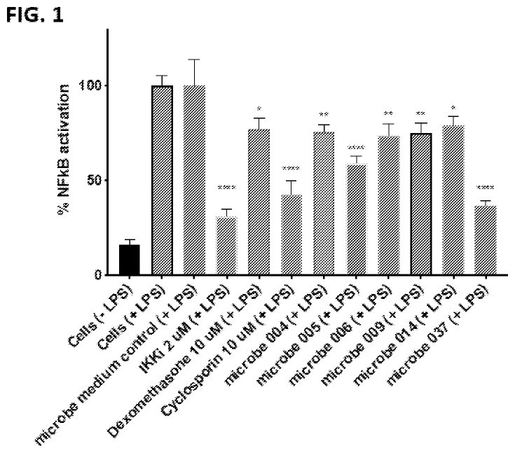

Supernatants from microbes 004, 005, 006, 009, 014, and 037 suppressed LPS-driven NFκB signaling in THP-1-DUAL™ cells (FIG. 1).

### Example 3. LPS-Induced TNFα Assessment

Primary human peripheral blood mononuclear cells (PMBCs) (Zen-Bio, STEMCELL) were thawed rapidly at 37° C. in a water bath. The cells were transferred to 50 mL conical tubes and the volume was brought to 10 mL with LYMPH-1 thawing medium (Zen-Bio). Cells were centrifuged at 1500 rpm for 10 min and the supernatants were removed. Cell pellets were resuspended in 1 mL LYMPH-1, followed by addition of fresh RPMI Media (Gibco) containing 10% heat-inactivated fetal bovine serum (FBS, Invitrogen). The cell number for each PBMC donor was assessed with a Nexcelom T4 Cell Counter. For experiments, PBMCs from each donor were seeded into 48 well tissue culture plates at a density of 5×10⁵ cells per well and placed overnight in an incubator at 37° C. for 20 hours. The next day, supernatants from microbes of Table 1, prepared as described in Example 1, a microbe growth medium control, or dexamethasone (InvivoGen: 10 uM) were thawed and added to PBMC cultures at various concentrations for 2 hours. After 2 hours, cells were left untreated or were stimulated with E. coli LPS (Sigma: 100 ng/mL). Stimulation was allowed to continue for 6 hours until cell culture supernatants were collected and transferred into 96 deep well plates and placed at −80° C. until further analysis.

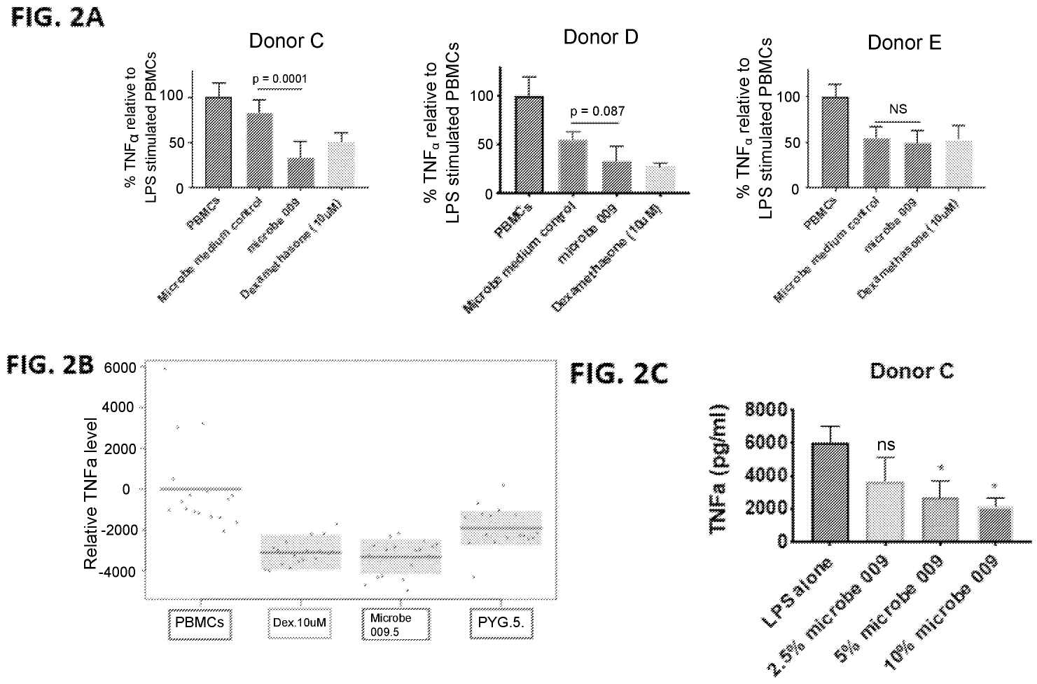

The dataset shown in FIG. 2B consisted of untreated baseline PBMCs, a positive control (dexamethasone), a bacterial media control (PYG), and PYG-based microbe supernatant from microbe 009. The data was collected across several donors. To explore whether the experimental conditions differed from untreated PBMCs, a mixed-effects model was fit, treating donor as random effects and using PBMCs as the contrast level in the regression. The resulting procedure pooled variance across all experimental conditions, and the contrast plot generated from this regression does not plot variance for the contrast level, which is considered the fixed point of comparison. There was modest heteroscedasticity for the data in the contrast level. The analysis shows that microbe 009 (Table 1) suppressed LPS-driven TNFα level in primary human PBMCs (FIG. 2A). Microbe 009 was significantly more suppressive than the bacterial media control (PYG) and had comparable effects to the dexamethasone control across 6 primary human PBMCs (FIG. 2B) and suppressed LPS-driven TNFα level in a dose-dependent manner (FIG. 2C).

To further confirm these conclusions, a separate all-pairs t-test comparison was performed, with corresponding correction for multiple hypothesis testing, and both dexamethasone and EMP-009 were statistically differentiated from the PYG negative control. Separate comparisons were performed for all donors, and the p-values were aggregated via Stouffer's Z method. Multiple comparisons were adjusted by Benjamini-Hochberg correction. The results for comparisons vs untreated PBMCs were consistent and about 10× less significant that for the above method, as would be expected given its greater statistical power, still showing statistically significant effects vs untreated level. Further, both dexamethasone and EMP-009 were also separated from bacterial media negative control.

### Example 4. LPS-Induced and TNFα-Induced NFκB Activity

A. Microbe Supernatant Pre-Treatment

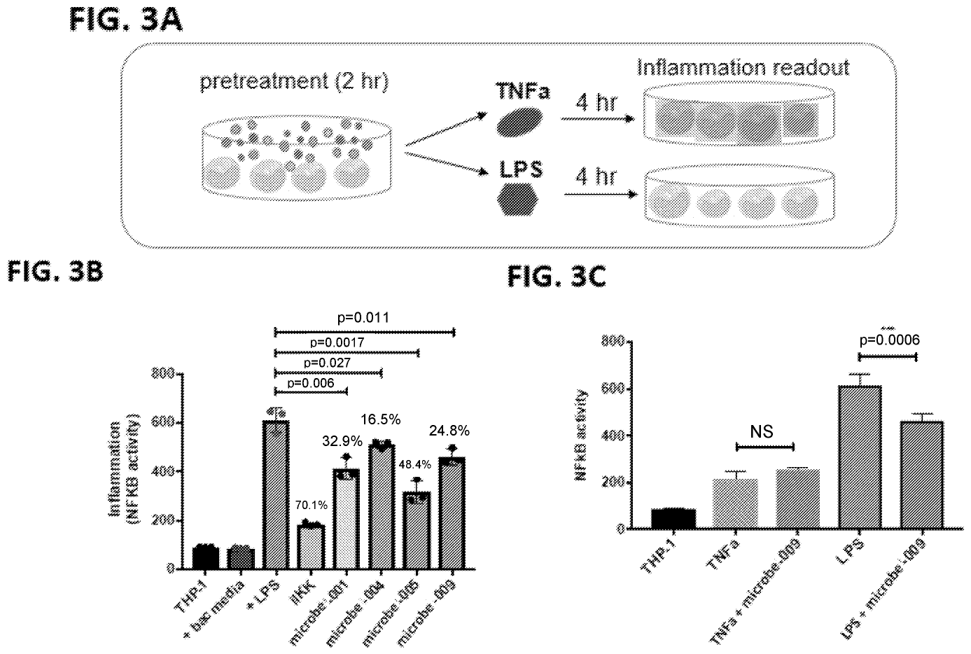

THP-1-DUAL™ cells were incubated with microbe supernatants (<3 KDa fraction, prepared as described in Example 1) for 2 hours, followed by TNFα or LPS challenge for 4 hours (FIG. 3A). NFκB activity was then assessed by measurement of secreted embryonic alkaline phosphatase) using QUANTI-Blue™ solution (InvivoGen). Microbial supernatants from microbes 001, 004, 005, and 009 (Table 1) reduced LPS-driven NFκB activation by 32.9%, 16.5%, 48.4%, and 24.8%, respectively, indicating that these microbial supernatants protected cells from LPS-mediated inflammation (FIGS. 3A and 3B). Supernatant of microbe 009 reduced LPS-driven NFκB activation, but had no effect on TNF-driven NFkB activation, indicating that the anti-inflammatory effect of microbe 009 is pathway-specific (FIGS. 3A-3C).

B. Treatment with Microbe Supernatant Following LPS or TNFα Challenge

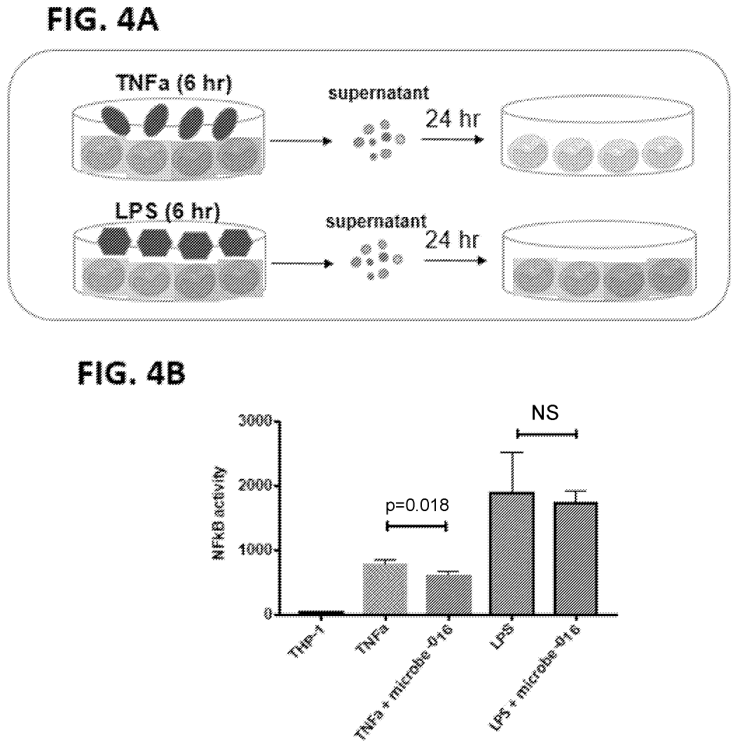

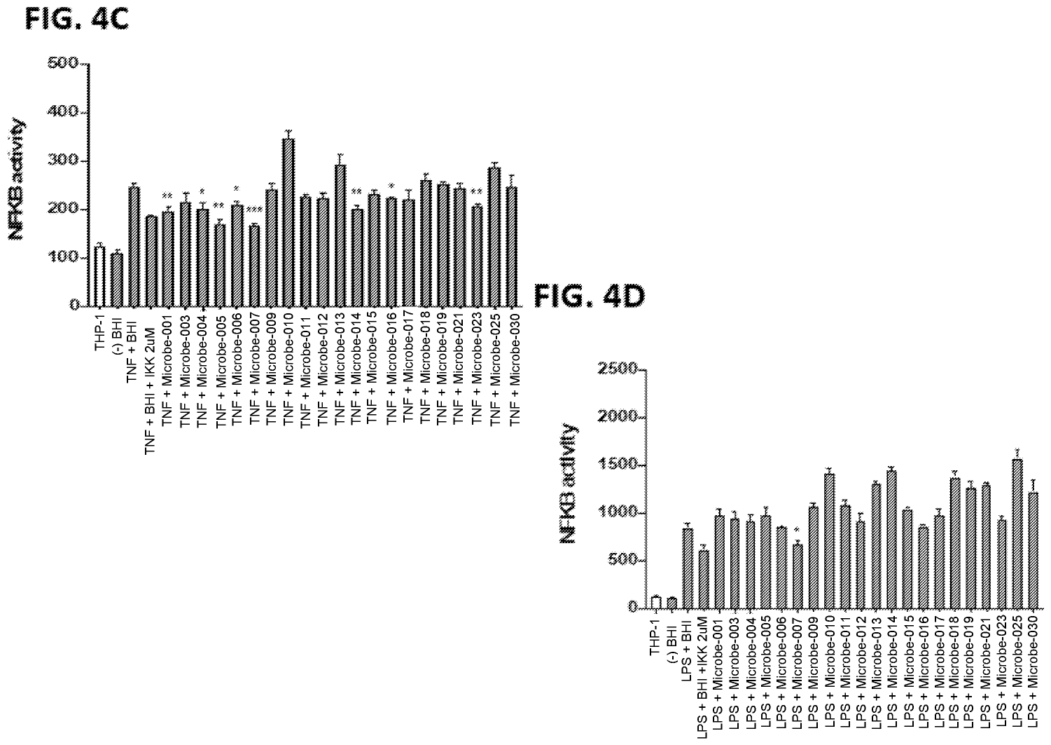

THP-1-DUALT™ cells were challenged with TNFα or LPS for 2 hours, followed by 24 hours (FIGS. 4A and 4B) or 4 hours (FIGS. 4C and 4D) of incubation with microbe supernatants (<3 KDa fraction) prepared as described in Example 1. NFκB activity was then assessed as described above (FIG. 4A). Supernatant of microbe 016 (Table 1) had a pathway-specific anti-inflammatory effect: TNFα-mediated NFκB activation was suppressed (˜22% reduction), but LPS-mediated NFκB activation was not affected.

Supernatant (<3 KDa fraction) from microbes 001, 004, 005, 006, 007, 014, 016, and 023 was found to suppress TNF-induced NFκB signaling (FIG. 4C). Supernatant (<3 KDa fraction) from microbe 007 was found to suppress LPS-induced NFκB signaling (FIG. 4D).

### Example 5. LPS-Induced Cytokine Assessment in PBMCs

Similar to the experiment described in Example 3 (above), PBMCs were seeded into tissue culture plates, rested for 24 hours and then pre-treated for 2 hours with 5% v/v of the <3 KDa fractions prepared from microbe-009 and microbe-005 supernatant or from unused bacterial growth media as control. Next, the cultures were stimulated with LPS (Sigma) at 100 ng/mL. 24 hours later, the supernatants were collected and cytokine responses were determined by bead based multi-plex array (LUMINEX® 200 Instrument System xMAP® Technology) according to the manufacturer's instructions.

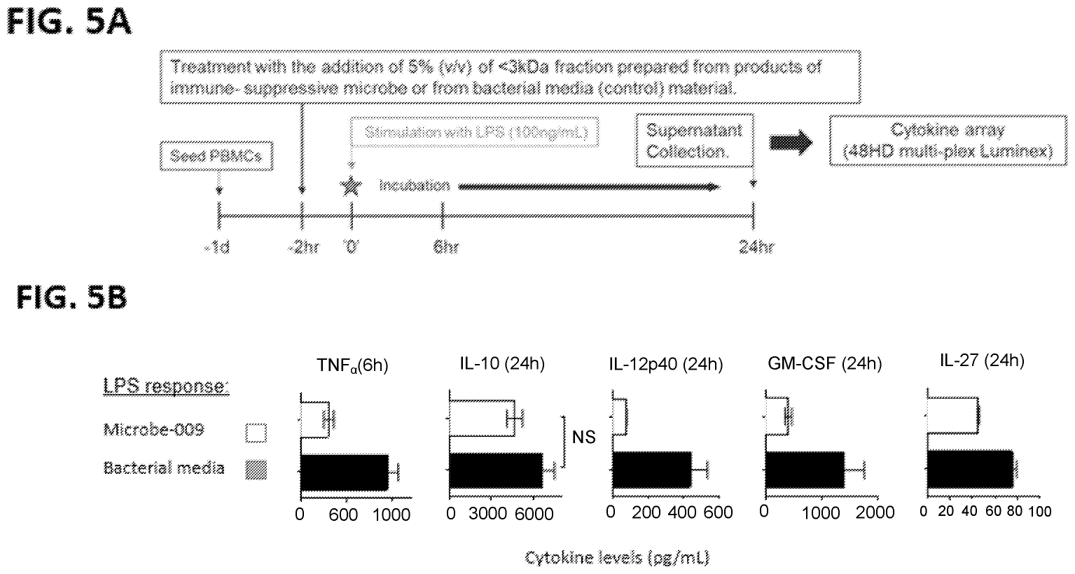

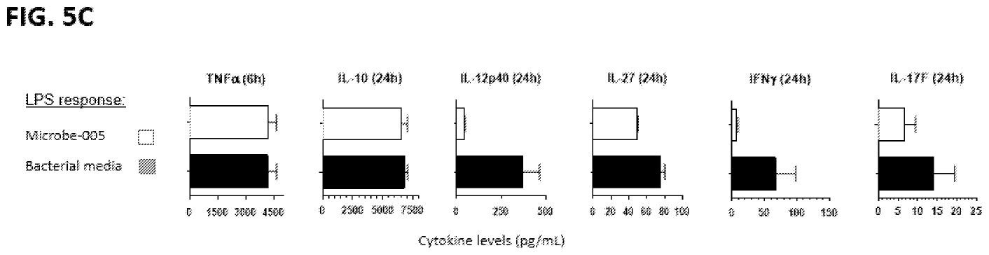

The data shown in FIGS. 5A-5C and in Tables 4 and 5 demonstrate the immune-modulatory effect of microbe-009 and microbe-005 products on cytokines expressed by PBMCs in response to stimulation with LPS. This example illustrates that fractions of microbial products purified from microbe-009 and microbe-005 selectively suppress key pro-inflammatory cytokines such as TNFα, IL-12p40 (part of IL-12 and IL-23), GM-CSF and IL-27, but have no significant effect on LPS-induced expression of IL-10, a key immune-modulatory cytokine.

TABLE 4
Cytokine suppression in response to treatment
with microbe-009 supernatant

| Cytokine | Suppression | Remarks |
|---|---|---|
| IL-12p40 | 83-85% | Biologically Significant |
| IL-27 | 40% | Reduction |
| TNFα | 50-60% | |
| GM-CSF | 71% | |
| IL-10 | NS | Biologically Insignificant |
| | | Reduction |

NS = not significant

TABLE 5
Cytokine suppression in response to treatment
with microbe-005 supernatant

| Cytokine | Suppression | Remarks |
|---|---|---|
| IL-12p40 | 90% | Biologically Significant |
| IL-27 | 45% | Reduction |
| IL-17F | 45-50% | |
| IFNγ | 90% | |
| TNFα | NS | Biologically Insignificant |
| IL-10 | NS | Reduction |

NS = not significant

### Example 6. Optimal Growth Media Conditions of Disease Associated Microbial Strains Determine Immune-Modulating Activity

A. Suppression of LPS Induced NFkB in Human Monocyte THP-1 Reporter Cells

THP1-BLUE™ NFkB, cells (InvivoGen; a reporter cell line comprising an NFkB-inducible secreted alkaline phosphatase (SEAP)) construct) were seeded in 96F tissue culture well plates (brain heart infusion (BHI) media, BHI-HK media (BHI media supplemented with Vitamin K₁ and Hemin), or Brucella blood agar (BRU-AS) media) at 25,000 cells/well. On the next day, 5% (v/v) of microbial products (Table 6) that were sub fractionated to <3 kDa size and Ethyl-Acetate (EtOAc) extracts were added to wells. After 2 hours of incubation, cells were stimulated with LPS (Sigma) at 100 ng/mL for 18 hours and supernatants were developed for assessment of NFkB reporter activity (SEAP) that was adjusted for cell number and viability according to the manufacturer's instructions. Data shown in Table 6 represent the relative change (%) and trend (+/−) in the mean LPS-induced NFkB activity in the treated cells. Assays were performed in triplicates. Controls for suppression (IKK 16 (Millipore Sigma) 2 μM inhibition) and inflammation are provided (Table 6). This example illustrates that a particular growth condition determines expression of immune-modulatory activity in disease associated microbial strains, demonstrated here with the LPS pathway using NFkB reporter. Further, this example illustrates that these conditions are different from one strain to another.

TABLE 6
NFkB reporter levels in LPS-challenged cells treated with microbial products

| Bacterial Strain # | Suppression of LPS induced NFkB activity in | | |
|---|---|---|---|
| (see Table 1) | THP1-Blue ™ cells (compared to untreated) | | |
| (EtOAc-fraction) | BHI | BHI-HK | BRU-AS |
| microbe-005 | −11% | (NS) | (NS) |
| microbe-009 | (NS) | −49% | −2% |
| microbe-016 | −1% | −6% | (NS) |
| microbe-017 | (NS) | −33% | (NS) |
| microbe-023 | (NS) | −39% | (NS) |
| microbe-030 | (NS) | −61% | (NS) |
| microbe-033 | (NS) | −30% | (NS) |
| microbe-038 | (NS) | −42% | −11% |
| microbe-041 | −3% | (NS) | −36% |
| microbe-043 | (NS) | −26% | −44% |
| microbe-045 | −8% | −9% | −17% |
| microbe-046 | −53% | −62% | −42% |
| microbe-047 | −12% | −44% | −2% |
| microbe-048 | (NS) | −44% | −17% |
| microbe-049 | −11% | −19% | −17% |
| microbe-050 | −12% | −35% | −49% |
| microbe-051 | (NS) | (NS) | (NS) |
| microbe-053 | (NS) | (NS) | (NS) |
| microbe-056 | −12% | (NS) | (NS) |
| microbe-057 | −10% | −26% | (NS) |
| microbe-058 | (NS) | −23% | (NS) |
| microbe-059 | −30% | −28% | (NS) |
| microbe-061 | −10% | −21% | (NS) |

| | | |
|---|---|---|
| Suppression > | IKK 16 | −80% |
| | 2 μM | |
| | inhibition | |
| Suppression > | Stimulation | 100% |
| | positive | |
| | control | |

NS: Not significant.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.
1. A method of treating or preventing an inflammatory disease or condition in a subject in need thereof, the method comprising administering to the subject a therapeutically effective amount of a composition comprising an isolated microorganism of Table 1, a lysate thereof, or a supernatant thereof
2. The method of paragraph 1, wherein the inflammatory disease or condition is an inflammatory bowel disease (IBD).
3. The method of paragraph 2, wherein the IBD is ulcerative colitis (UC).
4. The method of paragraph 2, wherein the IBD is Crohn's disease.
5. The method of paragraph 1, wherein the inflammatory disease or condition is an autoimmune disease or an allergy.
6. The method of paragraph 1 or 5, wherein the inflammatory disease or condition is selected from the group consisting of asthma, rheumatoid arthritis, dermatitis, psoriasis, psoriatic arthritis, multiple sclerosis, celiac disease, glomerulonephritis, hepatitis, and transplant rejection.
7. A method of treating or preventing a disease or condition modulated by an inflammatory pathway in a subject in need thereof, the method comprising administering to the subject a therapeutically effective amount of a composition comprising an isolated microorganism of Table 1, a lysate thereof, or a supernatant thereof
8. The method of paragraph 7, wherein the inflammatory pathway is an NFκB pathway or a TNFα pathway.
9. The method of paragraph 8, wherein the NFκB pathway is an LPS-driven NFκB pathway or a TNFα-driven NFκB pathway.
10. The method of any one of paragraphs 7-9, wherein activation of the inflammatory pathway is decreased by at least 20% relative to a baseline level in the treated subject or relative to a subject who has not been treated with the composition.
11. The method of any one of paragraphs 7-10, wherein the disease or condition is an IBD.
12. The method of paragraph 11, wherein the IBD is UC or Crohn's disease.
13. A method of modulating an inflammation marker in a subject in need thereof, the method comprising administering to the subject a therapeutically effective amount of a composition comprising an isolated microorganism of Table 1, a lysate thereof, or a supernatant thereof
14. The method of paragraph 13, wherein the inflammation marker is associated with an NFκB pathway or a TNFα pathway.
15. The method of paragraph 14, wherein the inflammation marker is NFκB, TNFα, TGFβ, IFNγ, IL12p40, IL-2, IL-4, IL-5, IL-6, IL-10, IL-13, IL-17, or a combination thereof
16. The method of paragraph 13 or 14, wherein the inflammation marker is IL1β, IL8, IL17A, or a combination thereof
17. The method of paragraph 13 or 14, wherein the inflammation marker is MCP-1, CCL20, NFAT, regulatory T cells (Tregs), Th17 induction, IL-23R, or IL-17A/F or a combination thereof
18. The method of paragraph 13 or 14, wherein the inflammation marker is M2-PK pyruvate kinase, osteoprotegerin, MPO, HMGB1, CHI3L1, HBD2, MMP, calprotectin, lactoferrin, pANCA, ASCA, or a combination thereof
19. The method of paragraph 13 or 14, wherein the inflammation marker is SAA, eotaxin-1, or a combination thereof
20. The method of any one of paragraphs 13-19, wherein the modulation is a decrease in the level of the inflammation marker.
21. The method of paragraph 20, wherein the decrease is at least 20% relative to a baseline level in the treated subject or relative to a subject who has not been treated with the composition.
22. The method of any one of paragraphs 13-19, wherein the modulation is an increase in the level of the inflammation marker.
23. The method of any one of paragraphs 1-22, wherein the composition comprises at least two isolated microorganisms of Table 1, lysates thereof, or supernatants thereof
24. The method of paragraph 23, wherein the composition comprises at least 3, 4, 5, 6, 7, 8, 9, 10, 15, 20, 25, 30, 35, 40, or 45 isolated microorganisms of Table 1, lysates thereof, or supernatants thereof
25. The method of any one of paragraphs 1-24, wherein the composition comprises one or more of said isolated microorganisms.
26. The method of paragraph 25, wherein the one or more isolated microorganisms colonizes the gut of the subject.
27. The method of any one of paragraphs 1-26, wherein the composition comprises a lysate of one or more of said isolated microorganisms.
28. The method of any one of paragraphs 1-27, wherein the composition comprises a supernatant of one or more of said isolated microorganisms.
29. The method of any one of paragraphs 1-28, wherein the isolated microorganism was grown in a culture medium selected from the group consisting of BHI media, BHI-HK media, and BRU-AS media.
30. The method of paragraph 29, wherein:
   (a) the isolated microorganism is microbe-005, microbe-056, or microbe-059 and the culture medium was BHI media;
   (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or
   (c) the isolated microorganism is microbe-041, microbe-043, microbe-045, or microbe-050 and the culture medium was BRU-AS media.
31. The method of any one of paragraphs 27-30, wherein the composition has been processed to remove endotoxin.
32. The method of any one of paragraphs 27-31, wherein the composition has been sterilized.
33. The method of any one of paragraphs 27-32, wherein the composition has been processed to remove all components having a molecular weight of more than 3 kDa.
34. The method of any one of paragraphs 27-32, wherein the composition has been processed to remove all components having a molecular weight of less than 3 kDa.
35. A composition comprising an isolated microorganism of Table 1, a lysate thereof, or a supernatant thereof, wherein the composition is formulated as pharmaceutically acceptable composition, a comestible composition, or a nutraceutical.
36. A composition comprising at least two isolated microorganisms of Table 1, lysates thereof, or supernatants thereof
37. The composition of paragraph 35 or 36, wherein the composition is formulated as a physiologically acceptable powder, granule, capsule, or tablet.
38. The composition of any one of paragraphs 35-37, wherein the composition is a pharmaceutical composition.
39. The composition of any one of paragraphs 35-37, wherein the composition is a comestible composition.
40. The composition of any one of paragraphs 35-37, wherein the composition is a nutraceutical.
41. The composition of paragraph 38, wherein the pharmaceutical composition is formulated for oral, enteral, or rectal administration.
42. The composition of paragraph any one of paragraphs 35-41, wherein the composition comprises at least 3, 4, 5, 6, 7, 8, 9, 10, 15, 20, 25, 30, 35, 40, or 45 isolated microorganisms of any one or more of Table 1, lysates thereof, or supernatants thereof
43. The composition of any one of paragraphs 35-42, wherein the composition comprises one or more of said isolated microorganisms.
44. The composition of paragraph 43, wherein the one or more isolated microorganisms colonizes the gut of a subject.
45. The composition of any one of paragraphs 35-44, wherein the composition comprises a lysate of one or more of said isolated microorganisms.
46. The composition of any one of paragraphs 35-45, wherein the composition comprises a supernatant of one or more of said isolated microorganisms.
47. The composition of any one of paragraphs 35-46, wherein the isolated microorganism was grown in a culture medium selected from the group consisting of BHI media, BHI-HK media, and BRU-AS media.
48. The composition of paragraph 47, wherein:
   (a) the isolated microorganism is microbe-005, microbe-056, or microbe-059 and the culture medium was BHI media;
   (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or
   (c) the isolated microorganism is microbe-041, microbe-043, microbe-045, or microbe-050 and the culture medium was BRU-AS media.
49. The composition of any one of paragraphs 45-48, wherein the composition has been processed to remove endotoxin.
50. The composition any one of paragraphs 45-49, wherein the composition has been sterilized.
51. The composition of any one of paragraphs 45-50, wherein the composition has been processed to remove all components having a molecular weight of more than 3 kDa.
52. The composition of any one of paragraphs 45-50, wherein the composition has been processed to remove all components having a molecular weight of less than 3 kDa.
53. A method of growing an isolated microorganism of Table 1, the method comprising culturing the isolated microorganism in a culture medium selected from the group consisting of BHI media, BHI-HK media, and BRU-AS media.
54. The method of paragraph 53, wherein:
   (a) the isolated microorganism is microbe-005, microbe-056, or microbe-059 and the culture medium is BHI media;
   (b) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium is BHI-HK media; or
   (c) the isolated microorganism is microbe-041, microbe-043, microbe-045, or microbe-050 and the culture medium is BRU-AS media.

Other embodiments are within the scope of the claims.

What is claimed is:

1. A method of treating or preventing an inflammatory disease or condition in a subject in need thereof, the method comprising administering to the subject a therapeutically effective amount of a composition comprising an isolated microorganism of Table 1, a lysate thereof, or a supernatant thereof.

2. The method of claim 1, wherein the inflammatory disease or condition is an inflammatory bowel disease (IBD).

3. The method of claim 2, wherein the IBD is ulcerative colitis (UC).

4. The method of claim 2, wherein the IBD is Crohn's disease.

5. The method of claim 1, wherein the inflammatory disease or condition is an autoimmune disease or an allergy.

6. The method of claim 1 or 5, wherein the inflammatory disease or condition is selected from the group consisting of asthma, rheumatoid arthritis, dermatitis, psoriasis, psoriatic arthritis, multiple sclerosis, celiac disease, glomerulonephritis, hepatitis, and transplant rejection.

7. A method of treating or preventing a disease or condition modulated by an inflammatory pathway in a subject in need thereof, the method comprising administering to the subject a therapeutically effective amount of a composition comprising an isolated microorganism of Table 1, a lysate thereof, or a supernatant thereof.

8. The method of claim 7, wherein the inflammatory pathway is an NFκB pathway or a TNFα pathway.

9. The method of claim 8, wherein the NFκB pathway is an LPS-driven NFκB pathway or a TNFα-driven NFκB pathway.

10. The method of any one of claims 7-9, wherein activation of the inflammatory pathway is decreased by at least 20% relative to a baseline level in the treated subject or relative to a subject who has not been treated with the composition.

11. The method of claim 7, wherein the disease or condition is an IBD.

12. The method of claim 11, wherein the IBD is UC or Crohn's disease.

13. A method of modulating an inflammation marker in a subject in need thereof, the method comprising administering to the subject a therapeutically effective amount of a composition comprising an isolated microorganism of Table 1, a lysate thereof, or a supernatant thereof.

14. The method of claim 13, wherein the inflammation marker is associated with an NFκB pathway or a TNFα pathway.

15. The method of claim 14, wherein the inflammation marker is NFκB, TNFα, TGFβ, IFNγ, IL12p40, IL-2, IL-4, IL-5, IL-6, IL-10, IL-13, IL-17, or a combination thereof.

16. The method of claim 13 or 14, wherein the inflammation marker is IL1β, IL8, IL17A, or a combination thereof.

17. The method of claim 13 or 14, wherein the inflammation marker is MCP-1, CCL20, NFAT, regulatory T cells (Tregs), Th17 induction, IL-23R, or IL-17A/F or a combination thereof.

18. The method of claim 13 or 14, wherein the inflammation marker is M2-PK pyruvate kinase, osteoprotegerin, MPO, HMGB1, CHI3L1, HBD2, MMP, calprotectin, lactoferrin, pANCA, ASCA, or a combination thereof.

19. The method of claim 13 or 14, wherein the inflammation marker is SAA, eotaxin-1, or a combination thereof.

20. The method of claim 13 or 14, wherein the modulation is a decrease in the level of the inflammation marker.

21. The method of claim 20, wherein the decrease is at least 20% relative to a baseline level in the treated subject or relative to a subject who has not been treated with the composition.

22. The method of claim 13 or 14, wherein the modulation is an increase in the level of the inflammation marker.

23. The method of any one of claims 1-5, 7-9, and 11-15, wherein the composition comprises at least two isolated microorganisms of Table 1, lysates thereof, or supernatants thereof.

24. The method of claim 23, wherein the composition comprises at least 3, 4, 5, 6, 7, 8, 9, 10, 15, 20, 25, 30, 35, 40, or 45 isolated microorganisms of Table 1, lysates thereof, or supernatants thereof.

25. The method of any one of claims 1-5, 7-9, and 11-15, wherein the composition comprises one or more of said isolated microorganisms.

26. The method of claim 25, wherein the one or more isolated microorganisms colonizes the gut of the subject.

27. The method of any one of claims 1-5, 7-9, and 11-15, wherein the composition comprises a lysate of one or more of said isolated microorganisms.

28. The method of any one of claims 1-5, 7-9, and 11-15, wherein the composition comprises a supernatant of one or more of said isolated microorganisms.

29. The method of any one of claims 1-5, 7-9, and 11-15, wherein the isolated microorganism was grown in a culture medium selected from the group consisting of BHI media, BHI-HK media, and BRU-AS media.

30. The method of claim 29, wherein:
(d) the isolated microorganism is microbe-005, microbe-056, or microbe-059 and the culture medium was BHI media;
(e) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or
(f) the isolated microorganism is microbe-041, microbe-043, microbe-045, or microbe-050 and the culture medium was BRU-AS media.

31. The method of claim 27, wherein the composition has been processed to remove endotoxin.

32. The method of claim 27, wherein the composition has been sterilized.

33. The method of claim 27, wherein the composition has been processed to remove all components having a molecular weight of more than 3 kDa.

34. The method of claim 27, wherein the composition has been processed to remove all components having a molecular weight of less than 3 kDa.

35. A composition comprising an isolated microorganism of Table 1, a lysate thereof, or a supernatant thereof, wherein the composition is formulated as pharmaceutically acceptable composition, a comestible composition, or a nutraceutical.

36. A composition comprising at least two isolated microorganisms of Table 1, lysates thereof, or supernatants thereof.

37. The composition of claim 35 or 36, wherein the composition is formulated as a physiologically acceptable powder, granule, capsule, or tablet.

38. The composition of claim 35 or 36, wherein the composition is a pharmaceutical composition.

39. The composition of claim 35 or 36, wherein the composition is a comestible composition.

40. The composition of claim 35 or 36, wherein the composition is a nutraceutical.

41. The composition of claim 38, wherein the pharmaceutical composition is formulated for oral, enteral, or rectal administration.

42. The composition of claim 35 or 36, wherein the composition comprises at least 3, 4, 5, 6, 7, 8, 9, 10, 15, 20, 25, 30, 35, 40, or 45 isolated microorganisms of any one or more of Table 1, lysates thereof, or supernatants thereof.

43. The composition of claim 35 or 36, wherein the composition comprises one or more of said isolated microorganisms.

44. The composition of claim 43, wherein the one or more isolated microorganisms colonizes the gut of a subject.

45. The composition of claim 35 or 36, wherein the composition comprises a lysate of one or more of said isolated microorganisms.

46. The composition of claim 35 or 36, wherein the composition comprises a supernatant of one or more of said isolated microorganisms.

47. The composition of claim 35 or 36, wherein the isolated microorganism was grown in a culture medium selected from the group consisting of BHI media, BHI-HK media, and BRU-AS media.

48. The composition of claim 47, wherein:
(d) the isolated microorganism is microbe-005, microbe-056, or microbe-059 and the culture medium was BHI media;
(e) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium was BHI-HK media; or
(f) the isolated microorganism is microbe-041, microbe-043, microbe-045, or microbe-050 and the culture medium was BRU-AS media.

49. The composition of claim 45, wherein the composition has been processed to remove endotoxin.

50. The composition of claim 45, wherein the composition has been sterilized.

51. The composition of claim 45, wherein the composition has been processed to remove all components having a molecular weight of more than 3 kDa.

52. The composition of claim 45, wherein the composition has been processed to remove all components having a molecular weight of less than 3 kDa.

53. A method of growing an isolated microorganism of Table 1, the method comprising culturing the isolated microorganism in a culture medium selected from the group consisting of BHI media, BHI-HK media, and BRU-AS media.

54. The method of claim 53, wherein:
(d) the isolated microorganism is microbe-005, microbe-056, or microbe-059 and the culture medium is BHI media;
(e) the isolated microorganism is microbe-009, microbe-016, microbe-017, microbe-023, microbe-030, microbe-033, microbe-038, microbe-046, microbe-047, microbe-048, microbe-049, microbe-057, microbe-058, or microbe-061 and the culture medium is BHI-HK media; or
(f) the isolated microorganism is microbe-041, microbe-043, microbe-045, or microbe-050 and the culture medium is BRU-AS media.

55. A composition of any one of claims 35 to 52 for use in a method of treating or preventing an inflammatory disease or condition in a subject in need thereof, wherein optionally the inflammatory disease or condition is an inflammatory bowel disease (IBD) (e.g., ulcerative colitis (UC) or Crohn's disease), is an autoimmune disease or an allergy, or is selected from the group consisting of asthma, rheumatoid arthritis, dermatitis, psoriasis, psoriatic arthritis, multiple sclerosis, celiac disease, glomerulonephritis, hepatitis, and transplant rejection.

56. A composition of any one of claims 35 to 52 for use in a method of treating or preventing a disease or condition modulated by an inflammatory pathway in a subject in need thereof, wherein optionally the inflammatory pathway is an NFκB pathway or a TNFα pathway, wherein further optionally the NFκB pathway is an LPS-driven NFκB pathway or a TNFα-driven NFκB pathway, and further wherein optionally activation of the inflammatory pathway is decreased by at least 20% relative to a baseline level in the treated subject or relative to a subject who has not been treated with the composition.

57. A composition of any one of claims 35 to 52 for use in a method of modulating an inflammation marker, e.g., as described herein, in a subject in need thereof.
